(19) United States
(12) Patent Application Publication
(10) Pub. No.: US 2008/0139916 A1
(43) Pub. Date: Jun. 12, 2008

(54) DETERMINING THE LENGTH OF A LONG, FLEXIBLE INSTRUMENT

(76) Inventors: Christian Maier, Munchen (DE); Claus Schaffrath, Munchen (DE)

Correspondence Address: RENNER, OTTO, BOISSELLE & SKLAR, LLP, 1621 Euclid Avenue, Nineteenth Floor, Cleveland, OH, 44115-2191, US

(21) Appl. No.: 11/935,571

(22) Filed: Nov. 6, 2007

Related U.S. Application Data
(60) Provisional application No. 60/866,077, filed on Nov. 16, 2006.

(30) Foreign Application Priority Data
Nov. 6, 2006 (EP) 06023012

(51) Int. Cl. A61B 19/00

(52) U.S. Cl. 600/407; 600/426

(57) ABSTRACT

A marker navigation system for determining a length of at least one flexible instrument having a first end and a second end, wherein portions of the at least one flexible instrument can be both linear and curved, the marker navigation system includes: a holder for holding the instrument, said holder comprising a first marker device, wherein the second end of the instrument is attached to the holder; a calibrating apparatus including a second marker device and at least one opening for inserting the first end of the instrument, said at least one opening having a predetermined shape and depth; a detection device for detecting a first location of the first marker device and a second location of the second marker device; and a data processing device for determining the length of the instrument. The data processing means determines the length of the instrument based on a) the detected first and second location; b) information concerning a cross-section of the instrument; c) information concerning an elasticity of the material forming the instrument; d) a relative location between the second end of the instrument and the first marker device; and e) a relative location between the second marker device and the at least one opening.

### RELATED APPLICATION DATA

This application claims priority of U.S. Provisional Application No. 60/866,077 filed on Nov. 16, 2006, which is incorporated herein by reference in its entirety.

### FIELD OF THE INVENTION

The present invention relates to determining the length of an instrument, such as a medical instrument.

### BACKGROUND OF THE INVENTION

In marker navigation systems, in particular in image-guided surgery (IGS), it is desirable to register and calibrate instruments, i.e., the location of parts of the instrument are made known relative to a reference system that, for example, is a spatial reference system or is associated with a detection means (e.g., a camera).

For some instruments, it is not possible or not desirable to attach a marker means to the tip of the instrument because if attached, the tip of the instrument (e.g., a needle) can not be used for its intended purpose. In this case, it is still desirable for the marker navigation system to provide information concerning the location of the tip of the instrument. To this end, information concerning the length of the instrument is required. For rigid instruments, the length of the instrument, for example, can be simply determined by tapping or otherwise identifying the two ends of the instrument with a pointer. The distance between the ends then corresponds to the length of the instrument. However, accuracy of the measurement suffers when the instrument is not a rigid instrument. The loss of accuracy increases as the elasticity of the instrument increases.

### SUMMARY OF THE INVENTION

Marker means are detected by a detection means (e.g., a camera, ultrasound detector, or the like). The marker means typically comprise three markers arranged in a fixed and predetermined location relative to each other, and may be mechanically connected to one another. The markers can be passive or active markers, wherein passive markers reflect signals (e.g., waves and/or radiation) emitted in their direction, and active markers are themselves the source of the signals (e.g., radiation and/or waves). The signals emitted from the (active or passive) markers, which, for example, can be wave signals or radiation signals, may be detected by the detection means (e.g., the camera). In order to establish a position of the marker means relative to the detection means, the marker means preferably is moved to provide the detection means with different views of the marker means. On the basis of these different views, the location of the marker means relative to the detection means can be determined in a known way, in particular in a spatial reference system. Reference is made in this respect to DE 196 39 615 A1 and the corresponding US publication 6,351,659, which are hereby incorporated by reference in their entireties.

The location of the marker means can be determined by the position of the marker means in a predetermined reference system. The reference system is preferably a reference system in which the detection means lies. The location of the marker means can be determined by the positions of the markers, in particular the center points of the markers in the reference system. The positions, for example, can be described using Cartesian coordinates or spherical coordinates. The location of one part (e.g., the detection means or marker means) relative to another part (e.g., a marker means) can be described by spatial angles, distances, coordinates (in a reference system) and/or vectors and is preferably calculated from the positions describing the location, for example by means of a program running on a computer.

The term “relative location” used here or the expression “location of a part A relative to a part B” thus comprises the concept of the relative positions between the two parts, such as between the marker means and/or their markers or between a marker means (or its markers) and the detection means. In particular, centers of gravity or center points of the parts can be selected as a punctiform reference point for establishing a position. If the position of one part is known in a reference system, then it is possible, based on the relative location of the two parts, to calculate the position of one of the two parts from the position of the other of the two parts.

If the marker means comprises only two markers, a start position is preferably known, and the marker system then allows the location of the marker means to be tracked when the marker means is spatially moved.

The marker means preferably comprises at least two markers, and more preferably three markers, and can of course also comprise more than three markers. The dimensions of the markers and the locations of the markers relative to each other are preferably known and available as prior-known data of a data processing means. The shape of the markers is preferably also known.

The marker navigation system preferably also comprises a detection means that detects signals from the at least two markers. As stated above, these are signals emitted from the markers (either actively emitted by the markers or are reflected by the markers). In the latter case, a signal transmitting source, for example an infrared light source, can be provided that emits signals (e.g., ultrasound waves or infrared light) towards the passive markers (continuously or in pulses), wherein the passive markers reflect the signals. A data processing means, such as a computer, allows the location of the marker means relative to the detection means to be calculated, in particular the location of the marker means in a reference system in which the detection means lies, e.g., in a reference system that lies in an operating theater.

The data processing means is preferably configured to perform calculating and/or determining operations. The data processing means can calculate the locations of the marker means based on the detected signals emitted from the marker means. The objects (a body structure or instrument) to which the marker means are attached are preferably calibrated. This means that the relative locations at least between parts of the object and the marker means attached to the object are known and/or stored in the data processing means, such that signals that describe the locations of the objects can be determined based on the locations of the marker means. The locations of the marker means are preferably calculated relative to the detection means, e.g., in a reference system in which the detection means lies. The locations can of course also be calculated in another reference system, e.g., in a reference system in which the patient lies and/or in which one of the marker means lies.

The marker navigation system allows the length, diameter and shape of the instrument to be determined. Additionally, the possible locations of the instrument also can be determined.

The marker navigation system comprises a holder that is designed to hold the instrument. One end of the instrument (the second end) can be detachably attached (mechanically) to the holder. The instrument can be attached to the holder such that the relative location between the second end of the instrument and the holder is fixed, i.e., the second end of the instrument is stationary relative to the holder. As stated above, this is not guaranteed for the other end (the first end) of the instrument, since the instrument is at least to a certain extent flexible. If the instrument were rigid, the first end of the instrument would also be stationary relative to the holder. The holder preferably comprises a handle and, for example, can comprise a machine such as a drilling apparatus that is designed to rotate the fixed instrument.

A calibrating apparatus also may be provided that allows the diameter and/or the cross-sectional shape of the instrument to be determined. To this end, at least one opening may be provided in the calibrating apparatus. If multiple openings are provided, then they preferably have different shapes, such that differently configured instruments can be inserted therein. Preferably, instruments having a cross-sectional shape that is constant over the entire length of the instrument are measured, since information concerning the shape of the instrument is preferably incorporated into calculating the length of the instrument and/or the possible locations of the first end of the instrument, e.g., the assumption that the cross-section of the instrument is the same over the entire length.

If multiple openings are provided in the calibrating apparatus, then the openings preferably have different shapes and sizes. In this way, one of the openings can be assigned to an instrument by inserting the instrument into the openings in an exact fit. Since the shape and size of the opening is known, the shape and size of the cross-section of the instrument corresponds to that of the opening. Further, since the location of the opening relative to the second marker means attached to the calibrating apparatus is also known, the location of the first end of the instrument is known or can be determined if the instrument has been inserted in an exact fit, and in particular all the way into the opening. Also, since the location of the base of each opening relative to the second marker means is also known, the location of the first end of the instrument is thus also known.

If an instrument has a circular cross-section, the at least one opening is also an opening having a circular cross-section, e.g., a cylindrical bore. Openings having other shapes, e.g., rectangular shapes, can of course also be provided to be able to insert in an exact fit and so measure instruments having a rectangular cross-section.

The aforesaid detection means can be configured to detect a first location of the first marker means, which the holder exhibits, and a second location of the second marker means, which the calibrating apparatus exhibits.

The aforesaid data processing means allows the length of the instrument to be determined, in particular calculated, using particular information that originates from and/or is predetermined by the detection means and/or is input into the data processing means. This information can include the following. The aforesaid first and second location (as determined by the detection means), and the data relating to these locations are input into the data processing means. The information concerning the cross-section of the instrument can be input by a user who reads off an identification number or the like that is written next to the opening into which the instrument has been inserted, wherein the identification number identifies the shape and/or size and/or diameter of the opening. Alternatively, this can of course also be achieved by image processing, wherein a camera (for example the same one used as the detection means) watches the region in which the first end of the instrument is inserted into the at least one opening of the calibrating apparatus. By optically evaluating the camera image (e.g., the markings next to the openings), it is then possible for the data processing means to ascertain which opening the first end of the instrument is inserted in.

Other possible ways of determining the opening in which the instrument has been inserted are disclosed further below in the detailed description. If the information concerning the cross-section (for example the diameter, cross-sectional area and/or shape of the cross-section) of the instrument is thus known, then one piece of information concerning the instrument is given. The other piece of information concerning the instrument, namely its length, can be determined, for example, based on information concerning the elasticity of the material of the instrument. The elasticity of the material of the instrument, for example, can be described by its elasticity modulus, also called Young's Modulus. The relative location between the second end of the instrument and the first marker means also can be used to determine the length, wherein it is possible to utilize the fact that the second end of the instrument is attached to the holder, abutting an abutment area of the holder. The relative location between the abutment area of the holder and the first marker means (of the holder) is preferably known and stored in the data processing means. “Known data” or “known information” or the like such as mentioned here are preferably data stored in the data processing means. The same applies to the relative location between the second marker means (of the calibrating apparatus) and the at least one opening, wherein as already stated above the relative location between the base of the at least one opening, which the first end of the instrument preferably abuts, and the second marker means is known.

In addition to the aforesaid information, a force that acts on the instrument is preferably also known. In this case, it can be assumed that the instrument is linear if there is no force acting on it, and extends in a curve if there is a force acting on it, i.e., it is bent by the action of the force.

When calculating, a constant curvature of the instrument may be assumed, e.g., no kinks are thought to arise in the longitudinal profile of the instrument. Alternatively or additionally, it can be assumed that the force acting on the instrument only acts on the ends of the instrument, preferably only at one end, wherein when calculating by means of the data processing means, it is preferably also assumed that at the end at which the force acts, it acts in the direction in which the instrument extends at this end, i.e., the direction of the force corresponds to the direction of a tangent applied to the curved instrument at the end of the instrument. In particular, a force acts between a front face of the instrument at the second end of the instrument and the holder, e.g., it is assumed that using the holder, a force is exerted on the front face of the instrument, said force resulting in a curvature of the instrument.

For determining the length of the instrument, the holder is preferably moved to detect a plurality of first and/or second locations, and these locations can be incorporated into the calculation. In this way, the precision with which the length of the instrument is determined can be increased, for example by forming an average value of the lengths determined for the different first and/or second locations. Alternatively or additionally, during use of the instrument a possible deviation of the instrument can be determined from the different detected first and second locations, and preferably stored by the data processing means. It is thus possible to establish which possible locations the first end can assume relative to the second end. It is then advantageous if the method for determining the length is performed by the same user that later uses the instrument, since it may be assumed that the user will apply similar forces. The possible relative locations between the first end and the second end thus established can then be indicated as possible locations during a (medical) application of the instrument attached to the holder. Alternatively, these possible locations of the instrument, in particular of the first end, also can be calculated as described below.

It is possible, once the length of the instrument has been successfully determined and without further using the calibrating apparatus, to calculate the profile of the instrument for a given force (e.g., for a given magnitude and direction of the force) and in particular to determine the location of the first end of the instrument, if only the location of the second end of the instrument is known or can be determined. As stated above, the location of the second end of the instrument, for example, can be determined by detecting the first marker means attached to the holder and using the known relative location between the first marker means and the second end of the instrument. The known configuration (cross-section and length) of the instrument, in conjunction with the known elasticity of the material of the instrument, then allows the curvature and in particular the location of the first end of the instrument and/or the profile of the instrument from the first end to the second end to be calculated. This calculated information can be at least partially indicated by a display. If a part of the instrument that includes the first end is not visible to the operator (surgeon), then calculating the possible profile of the instrument allows a region to be calculated in which the non-visible part of the instrument can be situated given a known or measured force. When the instrument is inserted into a body structure, the instrument, for example, can be gradually deviated from a linear extension and pass into a curved extension. This can result in the first end being situated in an undesirable region of the body structure. Indicating the possible range of location of the instrument enables the surgeon to decide whether or not such a risk exists and, for example, to verify the risk using additional examination measures (x-ray images).

As stated above, the force, alternatively or in addition to being assumed or estimated based on a probable value for the force, also can be measured. To this end, a force sensor can be provided in the holder (in particular in the vicinity of the second end of the instrument) that measures the magnitude and/or direction of the force exerted by the holder on the instrument and relays the information to the data processing means for further processing.

### BRIEF DESCRIPTION OF THE DRAWINGS

The forgoing and other features of the invention are herein after discussed with reference to the drawing.

FIG. 1 shows an exemplary marker navigation system in accordance with the invention.

FIG. 2 schematically shows an exemplary curvature of an instrument as the basis for determining the length in accordance with the invention.

FIG. 3 is a schematic diagram showing possible profiles of the instrument within a body structure.

### DETAILED DESCRIPTION

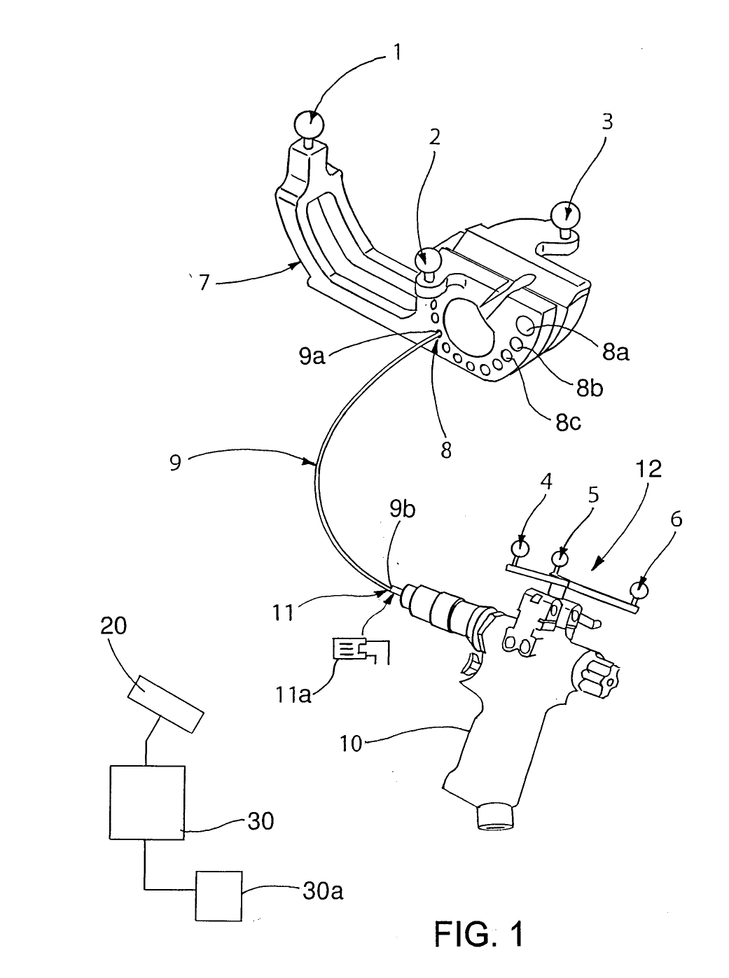

FIG. 1 shows a calibrating apparatus 7, an instrument 9, a holder 10, a detection means 20 and a data processing means 30. The calibrating apparatus 7 comprises a marker means having marker spheres 1, 2 and 3. The marker spheres 1, 2 and 3 are connected to each other via the body of the calibrating apparatus 7 and assume predetermined locations relative to each other. The body of the calibrating apparatus 7 preferably comprises openings. Of these openings, only the openings 8, 8a, 8b and 8c have been provided with reference signs, for the sake of clarity. The openings are preferably cylindrical recesses having a prior-known depth and diameter, and preferably differ with regard to their diameters. An identification number, which indicates the diameter of the opening, is preferably printed next to the opening.

The instrument 9 is preferably elongated and flexible, in particular elastic, and has a circular cross-section. In the image shown in FIG. 1, the instrument is shown significantly curved. In practice, however, the instrument can be designed to be significantly more rigid, only allowing a slight curvature. The instrument may be made of high-grade steel such as is used in medicine and surgery. The elasticity modulus of steel is around 190 GPa to 200 GPa. The elasticity modulus of other materials, such as for example glass fiber, is around 50 GPa to 90 GPa, whereas that of silicone rubber is for example only 10 MPa to 100 MPa.

The instrument also can exhibit other diameters, such as for example rectangular diameters, as in the case of a sheet metal plate. Examples of instruments include Kirschner wire, screws, in particular Schanz screws for bones, drills, intramedullary pins and Steinmann pins (for externally fixing the bone). Typical diameters are in the range of 0.1 mm or larger and/or 20 mm and smaller.

Other possible instruments are the shanks of a pair of forceps or the blades of a pair of scissors, which also exhibit a certain elasticity. Other examples of instruments are ultrasound probes, cannulae, catheters, in particular for ventricle drainage and for use in pain therapy (for example peridural anaesthesia, in particular peridural catheters) or needles such as are used in vertebroplasty or facet infiltration, for example.

The instrument 9 can be fastened to the holder 10 at an assembly end 11 of the holder 10. To this end, the instrument 9 can be inserted into a correspondingly designed sleeve provided at the assembly end 11 and connected, stationary, to the holder 10, for example due to the exact fit and/or by means of a screw. A marker means 12 also may be provided on the holder 10 and can include three marker spheres 4, 5 and 6. The marker spheres 4, 5 and 6 of the marker means 12 can be connected to each other via arms, such that they each assume a predetermined location relative to each other.

The calibrating apparatus 7 and the holder 10 are calibrated such that the relative locations of at least parts of the surface of the holder 10 and of the calibrating apparatus 7 are known relative to the marker spheres.

In particular, the relative location between the openings (8, 8a, 8b, 8c, . . . ) of the calibrating apparatus 7 is known relative to the marker spheres 1, 2 and 3. The relative location of the assembly end 11 relative to the marker means 12 (relative to the marker spheres 4, 5 and 6) is also known.

The data and information referred to above as “known” are preferably stored in the data processing means 30. The locations of the marker spheres 1, 2 and 3 relative to the openings and the locations of the marker spheres 4, 5 and 6 relative to the end 11 also can be stored in the data processing means 30. Data signals from the detection means 20 also may be provided to the data processing means 30. The detection means 20 detects signals from the marker spheres 1, 2, 3, 4, 5 and 6. The detection means 20 can be configured as a camera that detects light, for example infrared light, emitted from the marker spheres, in particular reflected by the marker spheres. The marker spheres reflect light if they are passive marker spheres. In this case, they can be irradiated by continuous or pulsed light sources, for example, such that they reflect the light.

The instruments used in conjunction with the invention are preferably thin. For example, the length can be a multiple of the diameter, e.g., more than twice or more than five times or more than ten times or more than twenty times the diameter of the instrument.

In the case described above, the geometry of the holder can be stored in the computer 30, e.g., the relative location between the marker means 12 and the assembly end 11 is known. Alternatively, the assembly end 11, for example, can be inserted into one of the openings 8, 8a, 8b or 8c in the calibrating apparatus until it comes into contact with the base of the holes. Since the depth and location (in particular the geometry) of the holes is known and the location relative to the marker spheres 1, 2 and 3 is known, it is possible for the data processing means 30 to calculate, from the signals detected by the detection means 20 and further processed by the data processing means 30, the location of the marker spheres 4, 5 and 6, which are also detected, relative to the assembly end 11.

The instrument 9 is inserted into an opening 8, preferably in an exact fit, such that the inner diameter of the opening 8 (at least approximately) corresponds to the outer diameter of the instrument 9. The diameter of the opening 8 can be read from the calibrating apparatus by an operator and input into the data processing means 30 for further processing.

Alternatively, the diameter of the instrument 9 also can be established automatically (without a manual input by a user). To this end, the openings 8, 8a, 8b, 8c, . . . ,for example, can be spaced far away from each other. The distance is in particular significantly greater than the deviation with which the first end 9a of the instrument 9 can deviate from a location that the first end 9aassumes when the instrument 9 is linear. As already mentioned above, only slight deviations of 1 cm, for example, may be expected when the instruments are made of steel. In this case, the aforesaid openings then can exhibit a distance of for example 2 cm. If this is the case, then the corresponding opening can be derived from the relative location between the marker means 12 and the calibrating apparatus 7. From this relative location, the data processing means 30 can determine that only one of the openings is possible as the opening into which the instrument 9 has been inserted. The other openings would require a significantly greater deviation of the instrument 9, which is not regarded as realistic. How the deviation of the instrument 9 (which is possible in practice) is calculated is outlined further below.

In addition to the information concerning the diameter of the instrument, the data processing means preferably also processes information concerning the elasticity of the material of the instrument. The elasticity modulus is preferably used to this end. The elasticity modulus is a material parameter from materials technology that describes the relationship between strain and distension when a solid body is deformed, given a linearly elastic behaviour. The greater the magnitude of the elasticity modulus, the more resistance a material offers against being deformed.

The location of the first end 9a of the instrument is also known, since said end 9a is inserted all the way into the corresponding opening 8. The location of the base of the opening 8 relative to the marker spheres 1, 2 and 3 also is known and stored in the data processing means 30. By detecting the marker spheres 1, 2 and 3, the location of the first end 9a of the instrument is thus known and can be further processed by the data processing means. The location of the abutment for the instrument in the sleeve 11 of the holder 10 relative to the marker means 12 is also known. The instrument 9 is also inserted all the way into the sleeve 11 (assembly end 11) of the holder 10. The location of the second end 9b relative to the marker means 12 is thus known. By detecting the marker means 12 by means of the detection means 20, the location of the second end 9b is thus known.

The following data are thus available in the data processing means 30 for further processing: the diameter of the instrument 9; the location of the ends 9aand 9b (for example relative to the detection means 20 in a reference system in which the detection means 20 lies); and the elasticity modulus of the material of the instrument. The length of the instrument 9 then can be calculated from the aforesaid known data as follows.

The elasticity modulus E allows the rigidity EI of a long, thin instrument having a circular cross-section to be calculated as follows:

$$EI = E * π \frac{{d}^{4}}{64} \qquad ( 1 )$$

The differential equation which describes the profile or curvature of the bent instrument is as follows:

$$\frac{{d}^{2} ϑ}{d {l}^{2}} - {γ}_{x} sin ϑ + {γ}_{y} cos ϑ = 0 \qquad ( 2 )$$

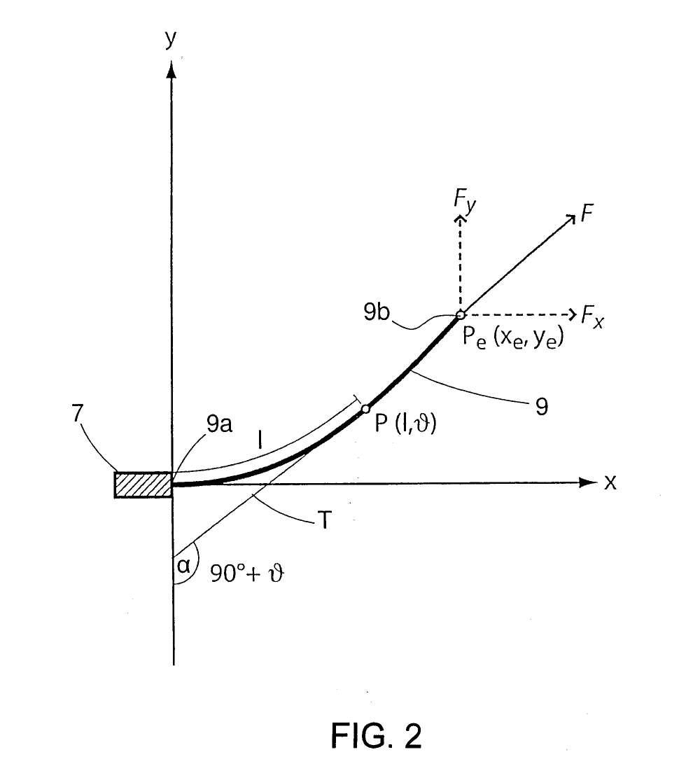

The angle Θ is explained in FIG. 2. The angle Θ results from applying the tangent to the curved instrument 9 at the second end 9b and the intersection point of this tangent T with the coordinate axis y, wherein for the obtuse angle α between the tangent T and the coordinate axis y, it holds that: α=90°+Θ. The origin of the x, y coordinate system corresponds to the base in the opening 8 in the calibrating apparatus 7, wherein the first end of the instrument abuts the base.

For Yₓ and Y_{y} mentioned in the above Equation (2), it holds that:

Yₓ=Fₓ/EI (2a)

and

Y_{y}=F_{y}/EI (2b)

As mentioned above, EI is the rigidity of the instrument, wherein E is the elasticity modulus and I is dependent on the geometry of the instrument and can be referred to as the cross-sectional moment of inertia. Rectangular cross-sections, for example, yield an I that deviates from that mentioned above in Equation (1). The variable “I” indicates the length from the first end 9a to a point P. The location of the point P can thus be described using the length I and the angle Θ.

The above Equation (2) can be integrated, and the following is then obtained:

$$\frac{1}{2} {( \frac{d ϑ}{d l} )}^{2} + {γ}_{x} cos ϑ + {γ}_{y} sin ϑ = c \qquad ( 3 )$$

wherein “c” is a constant and can be determined by the ancillary condition dΘ/dl=Y_{y}xₑ, which holds at the first end 9a of the instrument, i.e., at the origin of the coordinate system. At the point Pₑ, it holds that Θ=Θₑ, and the length of the instrument 9 can be calculated as follows:

$$L = {\int }_{0}^{{ϑ}_{e}} \frac{d ϑ}{\sqrt{2 {γ}_{x} + {γ}_{y}^{2} {x}_{e}^{2} - 2 {γ}_{x} cos ϑ - 2 {γ}_{y} sin ϑ}} \qquad ( 4 )$$

In the above equation, the variables Θₑ, Yₓ and Y_{y} are unknown. These three unknown variables can be calculated using the following conditions:

$$\begin{matrix}{x}_{e} = {\int }_{0}^{{ϑ}_{e}} \frac{cos ϑ d ϑ}{\sqrt{2 {γ}_{x} + {γ}_{y}^{2} {x}_{e}^{2} - 2 {γ}_{x} cos ϑ - 2 {γ}_{y} sin ϑ}} & ( 5 ) \\ {y}_{e} = {\int }_{0}^{{ϑ}_{e}} \frac{sin ϑ d ϑ}{\sqrt{2 {γ}_{x} + {γ}_{y}^{2} {x}_{e}^{2} - 2 {γ}_{x} cos ϑ - 2 {γ}_{y} sin ϑ}} & ( 6 ) \\ {γ}_{x} + \frac{1}{2} {γ}_{y}^{2} {x}_{e}^{2} - {γ}_{x} cos {ϑ}_{e} - {γ}_{y} sin {ϑ}_{e} = 0 & ( 7 )\end{matrix}$$

### The latter equation incurs the ancillary condition that the curvature of the instrument or more precisely the change in the angle Θ disappears at the point Pₑ, i.e., at Θ=Θₑ.

If, with the aid of the above equations (5), (6) and (7), the unknown variables Θₑ, Yₓ and Y_{y} have now been calculated, then the length of the instrument can be calculated with the aid of Equation (4).

In this calculation, it has been taken into account that the instrument bends in one plane. The coordinates in the plane are described using the Cartesian coordinate system comprising the x-axis and y-axis. Moreover, the points in the plane can be described using the coordinates Θ and I. It is also assumed that the instrument can freely rotate in the corresponding hole 8 in the calibrating apparatus 7, such that no additional forces, in particular torque forces, arise, i.e., it is assumed that the instrument 9 only bends in the xy plane and that no other deforming forces act on it.

The force that acts on the end 9b at the point Pₑ is the force F that can be broken down into two components Fₓ (i.e., the component acting in the x-direction) and F_{y} (i.e., the component acting in the y-direction). It is assumed that the direction of the force F corresponds to the tangent T described above. This is a realistic assumption, since the instrument 9 is in practice intended to be inserted, with the aid of the holder 10, for example, into a body structure, wherein a pressure force is exerted on the instrument 9 in the direction of the tangent T with the holder 10. This is done in order to advance the instrument 9 further in the body structure. It is thus assumed that no kinking forces and torques, which can result in the instrument 9 kinking at the assembly end 11 (i.e. at Pₑ), are exerted on the instrument 9.

Additionally, a force sensor 11a may be provided in the holding means 10. The force sensor is in particular in the vicinity of the assembly end 11. The force sensor 11a preferably measures the magnitude of the force acting between the instrument 9 and the holder 10. The dynamometer (force sensor 11a) in particular measures the magnitude of the force and preferably also the direction of the force. If the force sensor 11a is provided, then the force Fₓ and the force F_{y} can be determined. Yₓ and Y_{y} can thus be calculated from the above Equations (2a) and (2b). This simplifies the determination of the length L with the aid of Equations (4) to (7).

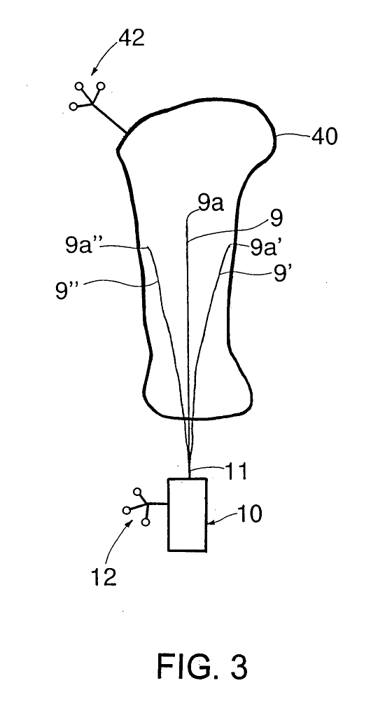

If the length of the instrument is now known, then this can be utilized when using the instrument to indicate possible positions of the first end of the instrument (assuming the position is unknown), e.g., the situation is assumed in which the second end is attached to the holder 10 and the instrument is inserted for example into a body structure by an operator (surgeon). The location of the first end is then unknown, but can be calculated as follows as described herein. The length L of the instrument is now a known variable. It is also assumed that a particular force acts in the direction of the longitudinal extension of the instrument, e.g., at an angle Θ=Θₑ. An example of a situation in practice in which the instrument 9 is inserted into a body structure 40 by means of the holder 10 is shown in FIG. 3. The instrument 9, for example a Kirschner wire, is inserted into a body structure (e.g. bone), wherein a typical force F is exerted in the longitudinal direction of the instrument 9. Typical forces, such as for example here form the basis in the above calculation, are for example greater than 0.1 N or 1 N or 10 N and for example smaller than 100 N or 1000 N. A typical force, for example, can thus be 1 N. This assumed force has the magnitude F and, for the aforesaid calculation, is broken down into the forces Fₓ and F_{y}, wherein Fₓ and F_{y} are a function of F and Θ. Equation (4) thus allows Θₑ to be calculated. Thus, the position Pₑ and therefore the relative location between the first end and the second end of the instrument then can be calculated via Equations (5) and (6). Since the location of the second end of the instrument can be determined by detecting the marker means 12, it is possible for the data processing means 30 to calculate from this the location of the first end of the instrument. Thus, this results in a possible bending of the instrument for a given force of magnitude F.

In the example shown in FIG. 3, the length of the instrument 9 is known and has for example been determined with the aid of the arrangement shown in FIG. 1. Thus, with the aid of the aforesaid equations, the possible location of the first end 9a of the instrument can be calculated. The possible locations are indicated in FIG. 3 as 9a, 9a′ and 9a″. The lines 9′ and 9″ indicate the possible profile of the instrument within the body structure 40. In the case shown in FIG. 3, it can thus be seen that if the instrument 9 is advanced further into the body structure 40, there exists a risk in that the tip of the instrument at the end 9a′ may exit the body structure 40 again, which may be undesirable.

The data processing means 30 is therefore preferably connected to a display 30a that, for example, shows the calibrated and registered body structure 40. The body structure 40, for example, and/or is connected to a marker means 42, a cone comprising possible positions of the first end 9a, 9a′ and 9a″ and/or possible profiles of the instrument in accordance with the lines 9, 9′ and 9″ from the first end to the second end of the instrument or a portion of the possible profiles. Thus, this enables the surgeon to assess the risk of the end 9a entering an undesirable region, without using an x-ray apparatus. In the case shown in FIG. 3, a verification of the location of the first end of the instrument 9 would thus be indicated, since the first end of the instrument is in danger of penetrating the outer skin of the bone 40 if the instrument 9 is advanced further. This is easily verified by the surgeon, since preferably both the body structure and possible locations of the instrument are shown by the device.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A marker navigation system for determining a length of at least one flexible instrument having a first end and a second end, wherein portions of the at least one flexible instrument can be both linear and curved, the marker navigation system comprising:
a holder for holding the instrument, said holder comprising a first marker device, wherein the second end of the instrument is attached to the holder;
a calibrating apparatus including a second marker device and at least one opening for inserting the first end of the instrument, said at least one opening having a predetermined shape and depth;
a detection device for detecting a first location of the first marker device and a second location of the second marker device; and
a data processing device configured to determine the length of the instrument while in a non-linear state based on
a) the detected first and second location,
b) information concerning a cross-section of the instrument,
c) information concerning an elasticity of the material forming the instrument,
d) a relative location between the second end of the instrument and the first marker device, and
e) a relative location between the second marker device and the at least one opening.

2. The marker navigation system according to claim 1, wherein the data processing device is further configured to determine the length of the instrument based on a predetermined or measured force that acts on the instrument so as to affect the shape of the instrument

3. The marker navigation system according to claim 2, wherein the predetermined or measured force acts on at least the first and/or second end of the instrument in a direction in which the instrument extends at the first and/or second end.

4. The marker navigation system according to claim 3, wherein the data processing device is further configured to calculate possible locations of the first end of the instrument when the location of the first end of the instrument is unknown, wherein the calculation is based on the force acting on the first and/or second end of the instrument and the determined length of the instrument.

5. The marker navigation system according to claim 4, further comprising a display operative to indicate the possible locations and/or a range of possible locations of the first end of the instrument, and/or indicate possible curvatures and/or a range of possible curvatures of the instrument.

6. The marker navigation system according to claim 2, wherein the data processing device is configured to calculate the length of the instrument based on the assumption that the instrument is deformed by the force into a shape comprising an arc.

7. The marker navigation system according to claim 1, wherein the information concerning the cross-section of the instrument includes information that the instrument has a circular cross-section of a particular diameter.

8. The marker navigation system according to claim 1, wherein the information concerning the elasticity of the material forming the instrument includes an elasticity modulus of the material.

9. The marker navigation system according to claim 1, wherein the information concerning the elasticity of the material forming the instrument includes information that the material is a high-grade steel.

10. The marker navigation system according to claim 1, wherein the instrument is a medical instrument.

11. A marker navigation system for determining a possible location within a body structure of at least one flexible instrument having a first end and a second end, wherein portions of the at least one flexible instrument can be both linear and curved, the marker navigation system comprising:
a holder for holding the instrument, said holder including a first marker device, wherein the second end of the instrument is attached to the holder;
a detection device for detecting a first location of a first marker device; and
a data processing device configured to determine the possible locations of the first end of the instrument in the body structure based on
a) the detected first location,
b) information concerning a cross-section of the instrument,
c) information concerning an elasticity of material forming the instrument,
d) a relative location between the second end of the instrument and the first marker device, and
e) a length of the instrument.

12. The marker navigation system according to claim 11, wherein the data processing device is further configured to determine the possible locations of the first end based on a predetermined or measured force that acts on the instrument so as to affect the shape of the instrument.

13. The marker navigation system according to claim 12, wherein the holder comprises a sensor operative to detect an action of the force created by the holder on the instrument, and the data processing device is configured to determine possible locations of the first end of the instrument based on the detected force.

14. The marker navigation system according to claim 11, wherein determining the possible locations of the instrument comprises at least one of:
determining possible locations of the first end of the instrument;
determining a profile of an extension of the instrument from the instrument's first end to the instrument's second end; or
determining a portion of the profile of the extension of the instrument.

15. The marker navigation system according to claim 11, wherein the instrument is a medical instrument.

16. The marker navigation system according to claim 11, wherein the data processing device is configured to output a range of possible locations of the first end of the instrument within the body structure.

17. A method for determining a length of a flexible instrument having a first end and a second end, wherein portions of the at least one flexible instrument can be both linear and curved, the method comprising:
attaching the instrument to a holder via the second end of the instrument, said holder comprising a first marker device;
inserting the first end of the instrument into an opening of a calibrating apparatus, wherein the calibrating apparatus comprises a second marker device and wherein a shape, depth and location of the opening relative to the second marker device are known;
detecting a first location of the first marker device and a second location of the second marker device via a detection device;
calculating the length of the instrument along a non-linear portion of the instrument using a data processing device configured to make said calculation based on
a) the detected first and second location,
b) information concerning a cross-section of the instrument,
c) information concerning an elasticity of the material forming the instrument,
d) a relative location between the second end of the instrument and the first marker device,
e) a relative location between the second marker device and the at least one opening.

18. The method according to claim 17, wherein calculating the length of the instrument further includes calculating the length based on a predetermined or measured force that acts on the instrument so as to affect the shape of the instrument.
